(19) United States
(12) Patent Application Publication
(10) Pub. No.: US 2013/0190546 A1
(43) Pub. Date: Jul. 25, 2013

(54) BENZENE ALKYLATION WITH STAGED ALKANE INJECTION

(71) Applicants: Feng Xu, Arlington Heights, IL (US); John Bauer, LaGrange Park, IL (US)

(72) Inventors: Feng Xu, Arlington Heights, IL (US); John Bauer, LaGrange Park, IL (US)

(73) Assignee: UOP LLC, Des Plaines, IL (US)

(21) Appl. No.: 13/357,342

(22) Filed: Jan. 24, 2012

(51) Int. Cl. C07C 2/66

(52) U.S. Cl. 585/470

(57) ABSTRACT

A process for making an alkylated aromatic is described. The alkylating agent is introduced into the reactor in at least two positions. This decreases the propane/benzene ratio at each injection point, improving the methylation reagent selectivity.

This invention relates generally to alkylating benzene, and more particularly to methods and apparatus for alkylating benzene having improved selectivity.

### BACKGROUND OF THE INVENTION

Alkylated aromatics include, among other compounds, the various isomers of xylene, i.e., ortho-, meta-, and para-xylene. Of these, para-xylene (p-xylene) is of particular value as a large volume chemical for the production of polyethylene terephthalate (PET), which is used, for example, as polyester fiber, film, and resin for a variety of applications. Because of downstream demand, the p-xylene market is robust and generally sees steady year-to-year demand growth in the range of about 6-8% per year.

Current processes for producing xylenes typically involve methylating toluene with methanol and/or olefins using a catalyst. Unfortunately, this approach has several challenges. First, methanol and olefins are relatively expensive reactants because they are in high demand for many other applications. In addition, the catalysts that are commonly used for these processes deactivate rapidly due to an excessive buildup of coke and heavy by-products on the catalyst during methylation. Catalysts also deactivate quickly due to hydrothermal de-alumination of the molecular sieves under process conditions because water is an unavoidable product from methanol. Finally, other less expensive methylation feed stocks, such as synthesis gas, and CO+H₂, have been reported, e.g., in U.S. Pat. No. 6,613,708, but the oxygenate reagents generate water as a side product, which undermines process efficiency and catalyst stability.

### SUMMARY OF THE INVENTION

One aspect of the invention is a process for making an alkylated aromatic. In one embodiment, the process includes introducing an aromatic feed into a reactor; introducing an alkylating agent into the reactor at two or more positions, the alkylating agent comprising an alkane, a cycloalkane, or combinations thereof; and reacting the aromatic feed with the alkylating agent under alkylating conditions to form the alkylated aromatic.

### BRIEF DESCRIPTION OF THE DRAWINGS

FIG. 1 illustrates one embodiment of an aromatic production complex including a benzene alkylation zone.

FIG. 2 illustrates one embodiment of a benzene alkylation zone.

FIG. 3 is a graph showing selectivity to methyl groups on phenyl rings vs. benzene conversion.

### DETAILED DESCRIPTION OF THE INVENTION

Reacting aromatic compounds, such as benzene, with alkylating agents, such as alkanes or C₅ to C₆ naphthenes, to make alkylated aromatics (e.g., toluene, xylenes, or trimethylbenzene) requires several performance indexes for the process to be efficient and cost effective. These include: benzene conversion (e.g., about 40% or more), phenyl ring retention (100% ideally), the highest possible methyl/phenyl ratio (0 in the feed and higher in the product) and high efficiency in utilizing alkane non-aromatic (NA) reagents (known as NA selectivity to alkylation).

It has been discovered that the alkane efficiency can be increased by introducing the alkylating agent in two or more points, as opposed to in a single injection. This can lead to increased benzene conversion per pass and increased alkylation yield. Dividing the alkylating agent into two or more portions and injecting them at different points along the catalyst bed decreases the propane to benzene ratio at each injection point. This improves the alkylation reagent selectivity to make alkylated aromatics. The amount injected at each point can be the same or different, and the distance between the injection points can be the same or different, as desired.

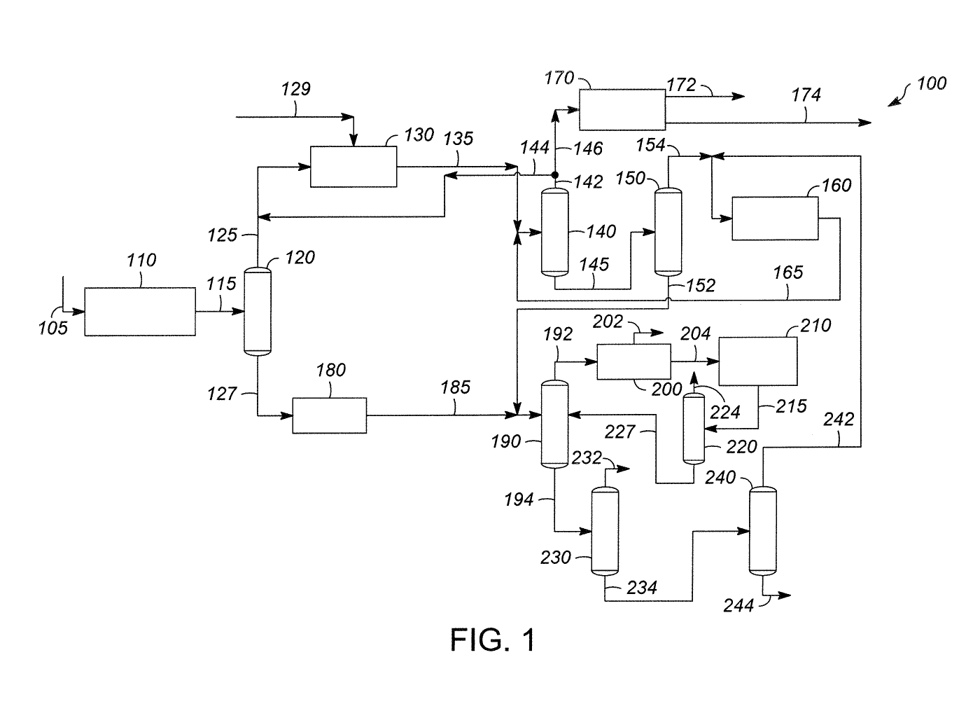

FIG. 1 illustrates one example of an aromatic production complex 100. The complex can permit the production of the desired aromatic product depending on market conditions. Particularly, utilizing the appropriate recycle streams, configurations, and feed amounts into various zones can allow the production of a desired product, such as xylenes, at the expense of benzene or vice-versa. Thus, the embodiments disclosed herein can provide flexibility depending on the desired aromatic to be produced. It can also provide the dealkylation of desired aromatics, such as the dealkylation of ethylbenzene.

The exemplary aromatic production complex 100 can include one or more reaction and separation zones, such as a naphtha hydrotreating and reforming zone 110, first fractionation zone 120, a benzene alkylation zone 130, a second fractionation zone 140, a third fractionation zone 150, a transalkylation zone 160, an extraction zone 170, a clay treatment zone 180, a fourth fractionation zone 190, a para-xylene separation zone 200, an isomerization zone 210, a fifth fractionation zone 220, a sixth fractionation zone 230, and an seventh fractionation zone 240. Although these zones are depicted in FIG. 1, it should be understood that additional reaction zones and/or fractionation zones may be included. At least some of these zones are disclosed in U.S. Pat. No. 7,601,311 B2 and U.S. Pat. No. 7,615,197 B2, as well as Robert A. Myers, Handbook of Petroleum Refining Processes, 3rd Edition, McGraw-Hill, 2003, Part II, pp. 2.3-2.63. In addition, it will be understood by those of skill in the art that FIG. 1 is illustrative only, and that other arrangements of the zones and their corresponding equipment are possible.

A naphtha feed stream 105 (e.g., containing hydrocarbon molecules having from about 5 to about 12 carbon atoms) is introduced to the hydrotreating and reforming zone 110. The hydrotreating and reforming zone 110 includes sub-zones for hydrotreating the naphtha feed stream 105 with a hydrotreating catalyst under hydrotreating conditions and for dehydrogenating and converting the hydrotreated naphtha, e.g., paraffins and/or naphthenes, with a reforming catalyst under reforming conditions to various aromatic compounds. The aromatic compounds, which preferably include benzene and toluene, are removed from the hydrotreating and reforming zone 110 in a reformate effluent 115 that also contains benzene co-boilers and heavy aromatics.

The reformate effluent 115 is introduced to the first fractionation zone 120 that separates the reformate effluent 115 into a first benzene-containing stream 125 and a first toluene-containing stream 127. The first benzene-containing stream 125 comprises C₆⁻ hydrocarbons, which includes benzene and the benzene co-boilers. There can also be small amounts of toluene and non-aromatic compounds. The first toluene-containing stream 127 comprises C₇⁺ hydrocarbons, which includes toluene, xylenes, ethyl benzene, and the heavy aromatics (e.g., C₉⁺).

The first benzene-containing stream 125 is introduced to the benzene alkylation zone 130. The benzene alkylation zone 130 contains a molecular sieve. As used herein, the term “molecular sieve” is defined as a class of adsorptive materials that are highly crystalline in nature, distinct from amorphous materials such as gamma-alumina. Various types of molecular sieves include aluminosilicate materials commonly known as zeolites. As used herein, the term “zeolite” in general refers to a group of naturally occurring and synthetic hydrated metal aluminosilicates, many of which are crystalline in structure. There are, however, significant differences between the various synthetic and natural materials, such as differences in chemical composition, crystal structure and physical properties. The zeolites occur as agglomerates of fine crystals or are synthesized as fine powders and are preferably tableted or pelletized for large-scale adsorption uses. Suitable zeolites include, but are not limited to, MFI, IMF, TUN, MSE, or MTW, and preferably MFI, which are described in The Atlas of Zeolite Structure Types by W. M. Meier. The alkylation reaction does not require a metal in the catalyst, although metals can be included for other purposes, if desired.

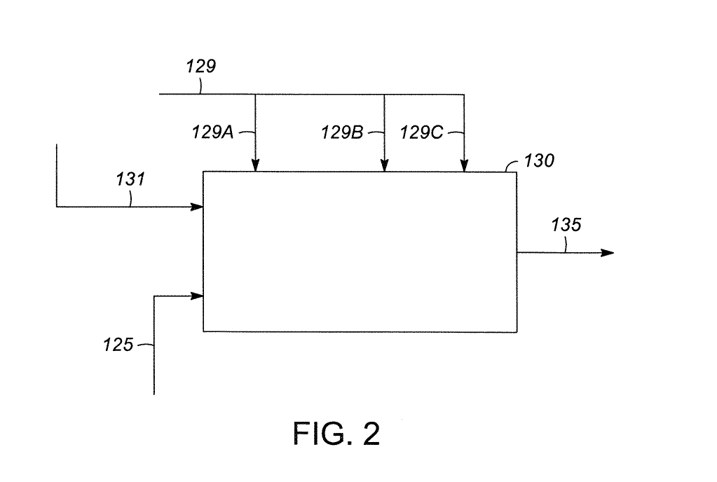

An alkylating agent stream 129 is introduced to the benzene alkylation zone 130. The alkylating agent stream 129 comprises paraffins and/or naphthenes. The alkylating agent stream 129 is introduced into the benzene alkylation zone 130 in two or more portions at different locations along the benzene alkylation zone 130, for example, at 2 positions, or 3, or 4, or 5, etc. As illustrated in FIG. 2, the alkylating agent stream 129 is injected at three positions 129A, 129B, and 129C. As shown, the distance between 129A and 129B is greater than the distance between 129B and 129C. However, the injection positions can be spaced apart equally, if desired. The amount of alkylating agent injected at each position can be the same or different, as desired.

In an exemplary embodiment, the alkylating conditions include a temperature of from about 350 to about 550° C., for example, from about 400 to about 500° C., a pressure of from about 345 to about 4,200 kPa, and a LHSV of from about 0.1 to about 50 hr⁻¹. Optionally, a hydrogen gas stream 131 can be introduced to the benzene alkylation zone 130. The ratio of hydrogen to hydrocarbon is typically in the range of 0 to about 4, or 0 to about 3, or 0 to about 2, or 0 to about 1. The ratio of aromatic to alkylation agent is generally in the range of about 10:1 to about 1:10, or about 2:1 to abut 4:1.

The alkylated aromatic-containing effluent 135 contains C₅⁻ hydrocarbons, and C₆⁺ aromatic hydrocarbons, such as benzene, toluene, xylene, and heavier aromatics. The alkylated aromatic-containing effluent 135 is passed to the second fractionation zone 140, where it is separated into a C₆⁻ hydrocarbon-containing stream 142 and a C₇₊ stream 145.

As illustrated, the C₆⁻ hydrocarbon-containing stream 142 is split into a first portion 144 that is combined with the first benzene-containing stream 125 and recycled to the benzene alkylation zone 130, and a second portion 146 that is sent to the extraction zone 170. The extraction zone 170 can utilize an extraction process, such as extractive distillation, liquid-liquid extraction, or a combination of liquid-liquid extraction/extractive distillation. The extraction zone 170 separates the second portion 146 of the C₆⁻ hydrocarbon-containing stream 142 into a raffinate stream 172, which contains the lighter end hydrocarbons (e.g., C₅⁻) and the non-aromatic benzene coboilers, and a benzene-rich stream 174.

The C₇₊ stream 145 is sent to the third fractionation zone 150 where it is split into a C₇₋ stream 154 and a C₈₊ stream 152. The C₇₋ stream 154 is sent to the transalkylation zone 160. The transalkylation zone 160 produces additional xylene and benzene with a transalkylation catalyst under transalkylation conditions via disproportionation and/or transalkylation reactions. The disproportionation reaction can include reacting two toluene molecules to form a benzene molecule and a xylene molecule, and the transalkylation reaction can include reacting toluene and a C₉ hydrocarbon to form two xylene molecules. Generally, the transalkylation catalyst is a metal stabilized transalkylation catalyst including a solid-acid component, a metal component, and an inorganic component, such as alumina. Typical transalkylation conditions can include a temperature of from about 200 to about 540° C., a pressure of from about 690 to about 4,140 kPa, and a LHSV of from about 0.1 to about 20 hr⁻¹. The effluent 165 from the transalkylation zone 160 can be combined with the alkylated aromatic-containing effluent 135 from the benzene alkylation zone 130 and sent to the second fractionation zone 140 for separation.

The first toluene-containing stream 127 from the first fractionation zone 120 can be sent to the clay treatment zone 180. The clay treatment zone 180 may include any suitable equipment for reducing olefins, such as a clay treater. The use of the clay treatment zone 180 typically depends on the content of the first toluene-containing stream 127.

The effluent 185 from the clay treatment zone 180 can be combined with the C₈₊ stream 152 from the third fractionation zone 150 and sent to the fourth fractionation zone 190. The fourth fractionation zone 190 separates the combined stream into C₈₋ stream 192 and C₈₊ stream 194 (which contains some o-xylene). The C₈₋ stream 192 is sent to the p-xylene separation zone 190 where a p-xylene rich stream 202 is removed for further processing.

The p-xylene-depleted stream 204 from the p-xylene separation zone 200 is sent to the isomerization zone 210 where additional p-xylene can be produced from various C₈ aromatic hydrocarbons by reestablishing an equilibrium or near-equilibrium distribution of the xylene isomers. The isomerized stream 215 from the isomerization zone 210 is sent to the fifth fractionation zone 220 where it is separated into a C₈₊ stream 227 which is sent back to the fourth fractionation zone 190 and a C₇₋ stream 224 which may be recovered or removed for processing. A portion of C₇₋ stream 224 can be added back into stream 129, if desired.

The C₈₊ stream 194 from the fourth fractionation zone 190 is sent to the sixth fractionation zone 230 where it is separated into an o-xylene rich stream 232 which is removed for further processing and a C₉₊ stream 234 which is sent to seventh fractionation zone 240.

The seventh fractionation zone 240 separates the C₉₊ stream 234 into a C₉₋ stream 242 and a C₁₀₊ stream 244. The C₉₋ stream 242 is sent to the transalkylation zone 160. The C₁₀₊ stream 244 is removed for further downstream processing and the like.

As used herein, the term “stream”, “feed”, “product”, “part” or “portion” can be used interchangeably and include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C₁, C₂, C₃ . . . Cₙ where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈ . . . Aₙ where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “alkylating agent” means a non-aromatic compound or radical used to produce higher alkyl substituted aromatic compounds. Examples of non-aromatic compounds include alkanes or cycloalkanes, preferably at least one C₂-C₈ alkane or C₅₊ cycloalkane. A non-aromatic radical can mean a saturated group forming a linear or branched alkyl group, a cycloalkyl, or a saturated group fused to an aromatic ring. Aromatic compounds having such non-aromatic radicals include cumene, indane, and tetralin. The alkylated aromatic compounds can include additional substituent groups, such as methyl, ethyl, propyl, and higher groups. Generally, an alkylating agent includes atoms of carbon and hydrogen and excludes hetero-atoms such as oxygen, nitrogen, sulfur, phosphorus, fluorine, chlorine, and bromine.

As used herein, the term “methylating agent” means a non-aromatic compound or radical used to produce higher methyl substituted aromatic compounds. Examples of non-aromatic compounds can include an alkane or a cycloalkane, preferably at least one C₂-C₈ alkane or C₅₊ cycloalkane. A non-aromatic radical can mean a saturated group forming a linear or branched alkyl group, a cycloalkyl, or a saturated group fused to an aromatic ring. Aromatic compounds having such non-aromatic radicals can include cumene, indane, and tetralin. The methylated aromatic compounds can include additional substituent methyl groups. Generally, an aromatic methylating agent includes atoms of carbon and hydrogen and excludes hetero-atoms such as oxygen, nitrogen, sulfur, phosphorus, fluorine, chlorine, bromine, and iodine. Such hetero-atom compounds may be referred to as a “methylating agent” and may include compounds such as iodomethane, dimethyl sulfate, dimethyl carbonate, and methyl trifluorosulfonate

As used herein, the term “radical” means a part or a group of a compound. As such, exemplary radicals can include methyl, ethyl, cyclopropyl, cyclobutyl, and fused ring-groups to an aromatic ring or rings.

As used herein, the term “rich” can mean an amount of at least generally about 50%, and preferably about 70%, by mole, of a compound or class of compounds in a stream.

As used herein, the term “substantially” can mean an amount of at least generally about 80%, preferably about 90%, and optimally about 99%, by mole, of a compound or class of compounds in a stream.

As used herein, the term “metal” can include rhenium, tin, germanium, lead, indium, gallium, zinc, uranium, dysprosium, thallium, chromium, molybdenum, tungsten, iron, cobalt, nickel, platinum, palladium, rhodium, ruthenium, osmium, or iridium.

As used herein, the methyl to phenyl ratio can be calculated as follows:

Methyl:Phenyl Mole Ratio=[Total number of methyls]/[Total Aromatic Rings]
Where: Total Aromatic Rings=sum over all i (MS (i)/MW(i)*NR(i))
Total Number of Methyls=sum over all i (MS (i)/MW(i)*ME(i))

### i: Compound Species

Molecular weight for species i: MW(i)
Number of aromatic (phenyl) rings for species i: NR(i)
Number of methyl groups attached onto the phenyl rings of species i: ME(i)
The mass content of species i, in the feed: MS (i)
Exemplary calculations for various compound species are depicted below:
Single ring aromatics: i: Toluene, NR(i)=1, ME(i)=1; is Xylene, NR(i)=1, ME(i)=2
Fused aromatic rings: i: Indane, NR(i)=1, ME(i)=0; is Tetralin, NR(i)=1, ME(i)=0; i: Naphthalene, NR(i)=1 ME(i)=0
Substituents on saturated fused ring: i: 1-methyl-indane and 2-methyl-indane (where one methyl group is attached to the five carbon ring), NR(i)=1, ME(i)=0
Substituents on unsaturated fused ring: i: 4-methyl-indane and 5-methyl-indane (where one methyl group is attached to the phenyl ring), NR(i)=1, ME(i)=1; is dimethyl 2,6-naphthalene, NR(i)=2, ME(i)=2

Hence, methyl groups are counted when attached to an aromatic group, e.g., phenyl, and not counted when attached to a full or partial, e.g., fused, saturated ring for fused-ring compounds having aromatic and saturated rings.

As used herein, the percent, by mole, of the aromatic ring recovery with respect to the feed can be calculated as follows:

Aromatic Ring Recovery=[Total Aromatic Rings, By Mole, of Product]/[Total Aromatic Rings, By Mole, of Feed]*100%

As used herein, the conversion percent, by weight, of C₆₊ non-aromatic compounds from the feed can be calculated as follows:

Conversion=(((Total Mass Feed C₆₊ non-aromatics−(Total Mass Product C₆₊ non-aromatics))/(Total Mass Feed C₆₊ non-aromatics))*100%

### Example

The following two tests demonstrate the yield and selectivities at a high and a low benzene/propane feed ratio. The examples are meant to illustrate the benefit of injecting propane at multiple points, which in effect increases benzene/propane ratios at two or more injection points, and thus promotes propane's carbon selectivity to forming methylated aromatics.

An MFI zeolite bounded with alumina phosphate (MFI (Si/Al₂=38)/AlPO₄ (67/33)) was in contact with mixed benzene and propane feeds having benzene/propane weight ratios of approximately 65/35 or 80/20. The same reaction conditions were used for both feed weight ratios: a pressure of about 2758 kPa (400 psig), H₂ co-fed at H₂/hydrocarbon mole ratio of 1, a WHSV of 2.5 and various temperatures.

As shown in Table 1, contacting the 65/35 feed required a lower temperature, 450° C., compared to 475° C. for the 80/20 feed, in order to reach the same 48-49% benzene conversion per pass. Contacting with a more diluted propane feed, i.e., 80/20 feed, makes more methylated aromatics and higher propane carbon selectivity to methyl-on-phenyl.

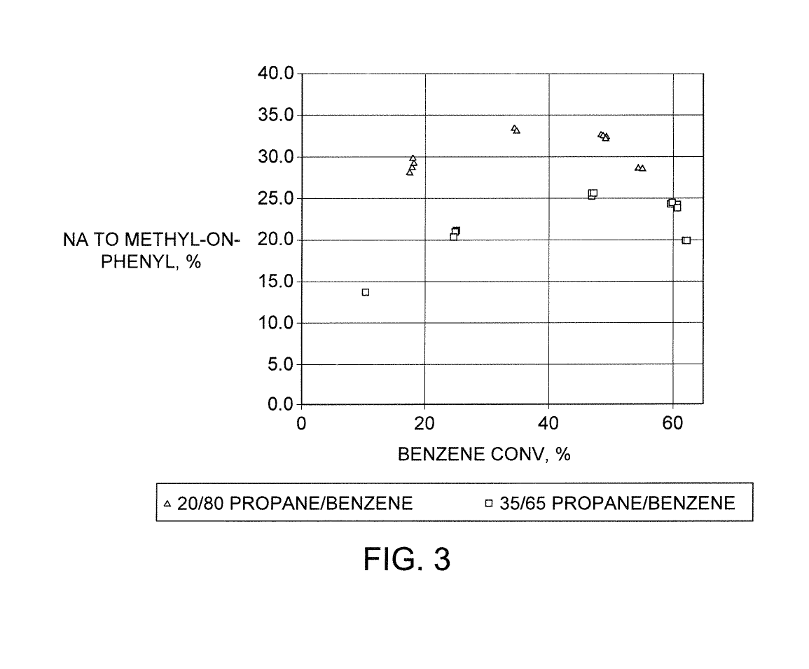

FIG. 3 shows that injecting propane at multiple points, which increases the benzene/propane ratio, has the benefit of better propane carbon selectivity to methyl-on-phenyl over wide benzene conversion ranges.

TABLE 1

| | 65/35 wt/wt | | | 80/20 wt/wt | | |
|---|---|---|---|---|---|---|
| Feed | Benzene/Propane | | | Benzene/Propane | | |
| Temp, ° C. | | 475 | 450 | | 475 | 450 |
| (setpoint) | | | | | | |
| Pressure, psig | | 400 | 400 | | 400 | 400 |
| Yield, wt % | Feed | Product | Product | Feed | Product | Product |
| C1 Yield | 0.1 | 12.6 | 7.6 | 0.1 | 7.8 | 4.5 |
| C2 Yield | | 12.2 | 7.3 | | 6.2 | 3.3 |
| C3 Yield | 34.1 | 5.0 | 13.9 | 20.5 | 3.4 | 9.2 |
| C4-5 non-A Yield | 0.1 | 0.5 | 1.6 | 0.2 | 0.3 | 0.8 |
| C6-C8 non-A Yield | | 0.0 | 0.0 | | 0.0 | 0.0 |
| Benzene | 65.7 | 25.0 | 33.0 | 79.2 | 40.3 | 51.8 |
| Toluene | | 27.7 | 22.9 | | 28.4 | 18.8 |
| Ethylbenzene | | 1.8 | 3.6 | | 2.1 | 4.6 |
| Xylene | | 10.0 | 6.1 | | 7.1 | 3.8 |
| C9 aromatics | | 3.4 | 2.7 | | 2.4 | 1.9 |
| C10+ (including | | 1.9 | 1.2 | | 2.0 | 1.3 |
| Naphthalenes) | | | | | | |
| Propane Conv, % | | 86 | 61 | | 84 | 56 |
| Benzene Conv, % | | 61 | 48 | | 49 | 35 |
| Methyl-on-Phenyl, | | 0.54 | 0.40 | | 0.48 | 0.30 |
| mole | | | | | | |
| Propane | | 24 | 26 | | 33 | 33 |
| selectivity to | | | | | | |
| Methyl-on-phenyl | | | | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for making an alkylated aromatic comprising:
introducing an aromatic feed into a reactor;
introducing an alkylating agent into the reactor at two or more positions, the alkylating agent comprising an alkane, a cycloalkane, or combinations thereof; and
reacting the aromatic feed with the alkylating agent under alkylating conditions to form the alkylated aromatic.

2. The process of claim 1 wherein an amount of the alkylating agent introduced at each position is the same.

3. The process of claim 1 wherein an amount of the alkylating agent introduced at each position is different.

4. The process of claim 1 wherein the alkylating agent is introduced in at least three positions, and wherein a distance between the first and second positions is the same as a distance between the second and third positions.

5. The process of claim 1 wherein the alkylating agent is introduced in at least three positions, and wherein a distance between the first and second positions is different from a distance between the second and third positions.

6. The process of claim 1 wherein the alkane comprises one or more C₂ to C₈ alkanes.

7. The process of claim 1 wherein the cycloalkane comprises one or more C₆₊ cycloalkanes.

8. The process of claim 1 wherein the aromatic feed comprises benzene.

9. The process of claim 1 wherein the aromatic feed is reacted with the alkylating agent in the presence of a catalyst.

10. The process of claim 1 wherein the aromatic feed comprises benzene and the alkylating agent is propane.

11. A process for making a methylated aromatic comprising:
introducing an aromatic feed into a reactor;
introducing a methylating agent into the reactor at two or more positions, the methylating agent comprising one or more C₂ to C₈ alkanes, one or more C₆₊ cycloalkanes, or combinations thereof; and
reacting the aromatic feed with the methylating agent under methylating conditions to form the methylated aromatic.

12. The process of claim 11 wherein an amount of the methylating agent introduced at each position is the same.

13. The process of claim 11 wherein an amount of the methylating agent introduced at each position is different.

14. The process of claim 11 wherein the methylating agent is introduced in at least three positions, and wherein a distance between the first and second positions is the same as a distance between the second and third positions.

15. The process of claim 11 wherein the methylating agent is introduced in at least three positions, and wherein a distance between the first and second positions is different from a distance between the second and third positions.

16. The process of claim 11 wherein the aromatic feed comprises benzene.

17. The process of claim 11 wherein the aromatic feed is reacted with the methylating agent in the presence of a catalyst.

18. The process of claim 11 wherein the aromatic feed comprises benzene and the methylating agent is propane.

19. A process for making a methylated aromatic comprising:
introducing benzene into a reactor;
introducing propane into the reactor at two or more positions; and
reacting the benzene with the propane under methylating conditions to form the methylated aromatic.

20. The process of claim 19 wherein the propane is introduced in at least three positions, and wherein a distance between the first and second positions is different from a distance between the second and third positions.
